(19) United States
(12) Patent Application Publication
(10) Pub. No.: US 2021/0072239 A1
(43) Pub. Date: Mar. 11, 2021

(54) BIOSENSOR COMPRISING LINKER MATERIAL AND QUANTUM DOT BEADS, AND TARGET ANTIGEN DETECTION METHOD USING SAME

(71) Applicant: ZEUS CO., LTD., Hwaseong-si, Gyeonggi-do (KR)

(72) Inventors: Heung Su JUNG, Yongin-si, Gyeonggi-do (KR); Sung Young SHIN, Osan-si, Gyeonggi-do (KR); Hyun Soo KIM, Yongin-si, Gyeonggi-do (KR); Sang Hyun PARK, Osan-si, Gyeonggi-do (KR); Ji Young LEE, Hwaseong-si, Gyeonggi-do (KR)

(73) Assignee: ZEUS CO., LTD., Hwaseong-si, Gyeonggi-do (KR)

(21) Appl. No.: 17/046,175

(22) PCT Filed: Apr. 19, 2019
(86) PCT No.: PCT/KR2019/004769

§ 371 Date: Oct. 8, 2020

(30) Foreign Application Priority Data
Apr. 23, 2018 (KR) 10-2018-0046848

(51) Int. Cl. G01N 33/558; G01N 33/553; G01N 33/533

(52) U.S. Cl. CPC G01N 33/558; G01N 33/533; G01N 33/553

(57) ABSTRACT

One aspect of the present disclosure relates to an immunochromatographic detection method for a target antigen in a biological sample, comprising the step of joining a linker having a first antibody and a quantum dot bead having a second antibody, with respect to the target antigen. By using the quantum dot beads and the linker, the method can successfully amplify detection strength and significantly increase detection sensitivity through a simple process without causing the loss of antigens participating in the detection when using only quantum dot beads. Furthermore, the present disclosure can significantly amplify detection strength without an additional washing step, thus enabling excellent detection and identification of a physiological substance in a biological sample, even in an actual product, and can be used to provide a product with a competitive price.

### CROSS-REFERENCE TO RELATED APPLICATIONS

This application is based upon and claims the benefit of priority from Korean Patent Application No. 10-2018-0046848, filed on Apr. 23, 2018, the entire contents of which are incorporated herein by reference.

### TECHNICAL FIELD

The present disclosure relates to a biosensor including a linker fluorescent substance and a quantum dot bead and a target antigen detection method using the same.

### BACKGROUND

In recent years, diseases have diversified, and the relationship between the physiological substances present in biological samples such as blood or urine and a disease or the physical condition of a subject is being widely studied and revealed. During this process, there has been the need for a technique which can rapidly, accurately and easily detect and identify a disease-related physiological substance in a biological sample.

As representative techniques for detecting a physiological substance, there are immunoassay techniques using biomarkers for a physiological substance such as enzyme-linked immunosorbent assay (ELISA), radioimmunoassay (RIA) and western blotting, etc. However, these techniques are complicated, time-consuming and costly, and need a lot of manpower. On the other hand, lateral flow immunoassay is a sandwich immunoassay technology using nanoparticles, which can easily and rapidly detect an analyte from a biological sample and have a low production cost, and thus has been primarily used in the diagnostic test field for a long time.

Fluorescent substances which are generally used in lateral flow immunoassays are gold nanoparticles that form immunocomplexes with physiological substances and develop a red color by a unique plasmon phenomenon. Due to these characteristics, these fluorescent substances have the advantage of easily detecting and identifying the presence or absence of a physiological substance from an actual product with the naked eye.

However, when gold nanoparticles are used, since the detection depends on a visual assessment, the sensitivity is not excellent, and analytical sensitivity is low, and thus the gold nanoparticles are mainly applied to physiological substances present in an excessive amount in blood. Accordingly, due to the difficulty of detecting or measuring a physiological substance present at a very low concentration in blood, there is a limit to early diagnosis of a disease. In addition, there is a problem in that a quantitative analysis of the physiological substances is difficult.

Therefore, to detect a low concentration of a physiological substance, efforts to amplify the detection strength of a fluorescent substance used in lateral flow immunoassay have continued. As one of such efforts, International Patent Publication No. WO 2008-071345 discloses that gold nanoparticles are stacked using a nucleotide complementary to a colloidal gold nanoparticle, thereby amplifying their fluorescence intensities.

However, according to the above technique, gold nanoparticles having complementary nucleotides may combine to each other before binding with a physiological substance such as an antigen, and when the gold nanoparticles are added simultaneously, they agglomerate. Such an agglomeration phenomenon disturbs the flow of a biological sample in lateral flow immunoassay, making the detection of a target physiological substance difficult. To prevent this phenomenon, before the injection of gold nanoparticles having different nucleotides, a washing step of removing conventionally existing nanoparticles is necessary. Therefore, to be applied to an actual lateral flow sensor, a washing step is required before new gold nanoparticles are added to the sensor, and thus the above technique has limitations in application to an actual sensor.

Therefore, the inventors of the present disclosure provide a detection method using a linker and a quantum dot bead as a technique for stably and very remarkably amplifying the detection fluorescence intensity in lateral flow immunoassay without a separate washing step.

### REFERENCE DOCUMENTS

1. US2010-0068727 A1

2. WO2008-071345 A1

### SUMMARY

Various embodiments of the present disclosure provide an immunochromatographic detection method for significantly improving sensitivity in a method of detecting a physiological substance by significantly amplifying the detection intensity using a very simple method without a separate washing step, and a diagnostic method or lateral flow immunosensor using the same.

An immunochromatographic detection method for a target antigen in a biological sample according to one aspect of the present disclosure may include binding a linker having a first antibody to a quantum dot bead having a second antibody through the target antigen.

According to one aspect of the present disclosure, the immunochromatographic detection method may be used in a method of diagnosing a target antigen-related disease, disorder or condition, a lateral flow immunosensor for detecting a physiological substance, and a bio-diagnostic kit.

According to the present disclosure, in some embodiments, an immunochromatographic detection method can very remarkably amplify the detection intensity and significantly improve detection sensitivity by a simple method without an antigen loss that occurs when a quantum dot bead is individually used, using a quantum dot bead and a linker.

The immunochromatographic detection method according to one aspect of the present disclosure can also exhibit the effect of significantly amplifying the detection intensity without a separate washing step, thereby rapidly and easily detecting and identifying a physiological substance in a biological sample during actual commercialization, which is advantageous in terms of competitiveness in pricing.

### BRIEF DESCRIPTION OF THE DRAWINGS

FIG. 1 is a schematic diagram showing the state in which a quantum dot, which is an example of a linker having a first antibody and a quantum dot bead having a second antibody are linked through binding with an antigen, which is a physiological substance, in a biological sample, so that the detection intensity is amplified in an immunochromatographic detection method according to one aspect of the present disclosure.

FIG. 2 is a graph showing the zeta potential of a quantum dot used in an immunochromatographic detection method according to one aspect of the present disclosure.

FIG. 3 is a graph showing the quantum efficiency of a quantum dot and a quantum dot bead, which may be used in an immunochromatographic detection method according to one aspect of the present disclosure.

FIG. 4 shows a transmission electron micrograph (FIG. 4A) of quantum dots and scanning electron micrographs (FIG. 4B) of quantum dot beads, which are used in an immunochromatographic detection method according to one aspect of the present disclosure.

FIG. 5 is a graph showing a particle analysis result for quantum dot beads used in an immunochromatographic detection method according to one aspect of the present disclosure.

FIG. 6 is a graph showing fluorescence intensities when quantum dots and quantum dot beads are individually used as a comparative example, and when quantum dots, which are one example of a linker, are used with quantum dot beads as an example in an experimental example of the present disclosure.

FIG. 7 is a schematic diagram showing a bio-diagnostic apparatus according to one aspect of the present disclosure.

FIGS. 8A to 8C are schematic diagrams showing various arrangements of pads present in a bio-diagnostic apparatus according to one aspect of the present disclosure.

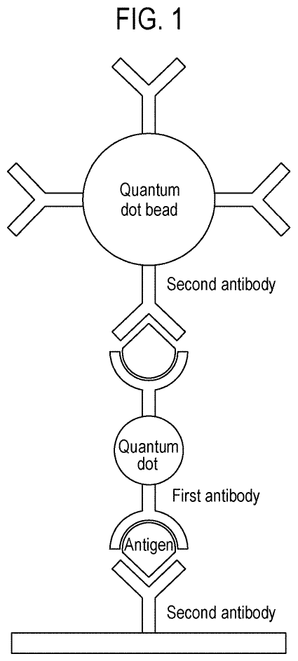

### DETAILED DESCRIPTION

In one aspect of the present disclosure, a “quantum dot” refers to a semiconductor nanoparticle, and has the characteristic of emitting different colors of light according to the size of the particle due to a quantum confinement effect. A quantum dot is approximately 20-fold brighter than a fluorescent dye such as a representative fluorescent substance, fluorescent rhodamine, and is approximately 100-fold more stable against photo-bleaching and has an approximately three-fold narrower spectral line width.

In one aspect of the present disclosure, a “quantum dot bead” is a particle including a large number of quantum dots, and is a broad concept that refers to all particles exhibiting the characteristic of being at least 100-fold brighter than a quantum dot and prepared to include multiple quantum dots regardless of the type of core constituting the quantum dot bead.

In one aspect of the present disclosure, a “linker” is to mediate the amplification of detection intensity by a quantum dot bead, and a broad concept that refers to all nanoscale particles capable of binding to an antibody. The linker may be a fluorescent substance, and when the linker is a fluorescent substance, it may further amplify the fluorescence detection intensity with a quantum dot bead.

In one aspect of the present disclosure, an “antigen” or “target antigen” is a physiological substance present in a biological sample, and a broad concept that includes all materials to be detected in connection with various diseases or the physical conditions of subjects. For example, in one aspect of the present disclosure, an antigen is a substance causing an immune response in a commonly referred biological sample, and includes all microorganisms, viruses, etc.

In one aspect of the present disclosure, a “biological sample” is a concept encompassing all samples having a physiological environment in which an antigen can be present, for example, urine, blood, serum, plasma, and saliva.

In one aspect of the present disclosure, an “antibody” is a broad concept that includes molecules inducing an immune response specifically against an antigen and binding to it so as to detect and identify the antigen. In addition, a “first antibody” and a “second antibody” recognize different epitopes of the same antigen, and is a broad concept that encompasses molecules present in pairs for antigen detection. For example, the second antibody may be fixed to a membrane of a diagnostic device to capture an antigen present in a biological sample, and the second antibody may have a detectable marker, rebind to the antigen captured by the second antibody to detect and identify the presence of the antigen in the biological sample.

In one aspect of the present disclosure, a “diameter” may refer to the length of the longest line segment passing through the center of a linker, a quantum dot or a quantum dot bead, and the average diameter may refer to the average of 10 line segments crossing the center, and in the case of the quantum dot, the diameter may refer to the size of a core-stable layer-shell layer or the size of a core-stable layer-shell-water soluble ligand layer.

Hereinafter, the present disclosure will be described in detail.

In one aspect of the present disclosure, an immunochromatographic detection method for a target antigen in a biological sample, which includes binding a linker having a first antibody to a quantum dot bead having a second antibody through the target antigen, may be provided.

In one aspect of the present disclosure, the first antibody and the second antibody may be specific for different sites, that is, different epitopes, of the target antigen.

In one aspect of the present disclosure, the linker may form a complex by binding to the antigen before binding to the quantum dot bead.

In one aspect of the present disclosure, the linker may be a substance that can bind to an antibody. Specifically, in one aspect of the present disclosure, the linker may be one or more selected from the group consisting of quantum dots, colloidal gold nanoparticles, colloidal carbon, colloidal selenium, up-conversion fluorescent nanoparticles, europium (III) chelate microparticles, dye-doped nanoparticles, magnetic nanoparticles, electroactive nanoparticles, silica, alumina, titanium dioxide, zinc dioxide, polystyrene, and polymethylmethacrylate, but the present disclosure is not limited thereto. More specifically, in one aspect of the present disclosure, the linker may be a quantum dot.

In one aspect of the present disclosure, the average diameter of the linker may be 1 to 300 nm, or 1 to 100 nm. Here, the average diameter of the linker may correspond to the range of all integers within the above range. Specifically, the average diameter of the linker may be 1 nm or more, 5 nm or more, 10 nm or more, 20 nm or more, 50 nm or more, 70 nm or more, 100 nm or more, 130 nm or more, 150 nm or more, 170 nm or more or 200 nm or more, or 300 nm or less, 280 nm or less, 260 nm or less, 240 nm or less, 220 nm or less, 200 nm or less, 180 nm or less, 160 nm or less, 140 nm or less, 120 nm or less, 100 nm or less, 80 nm or less, 60 nm or less, 40 nm or less, 30 nm or less, 20 nm or less or 15 nm or less. In one aspect of the present disclosure, the quantum dot may include one included in a quantum dot bead and one functioning as a linker.

In one aspect of the present disclosure, the quantum dot included in a quantum dot bead and the quantum dot functioning as a linker may have a core-stable layer-shell-water soluble ligand layer structure.

In one aspect of the present disclosure, the core may include one or more of cadmium (Cd) and selenium (Se); the stable layer may include one or more of cadmium (Cd), selenium (Se), zinc (Zn) and sulfur (S); and the shell may include one or more of cadmium (Cd), selenium (Se), zinc (Zn) and sulfur (S).

In one aspect of the present disclosure, the quantum dot may include one or more of Group 12 to 16 element-based compounds, Group 13 to 15-element-based compounds and Group 14 to 16 element-based compounds.

In one aspect of the present disclosure, the Group 12 to 16 element-based compounds include one or more of cadmium sulfide (CdS), cadmium selenide (CdSe), cadmium telluride (CdTe), zinc sulfide (ZnS), zinc selenide (ZnSe), zinc telluride (ZnTe), mercury sulfide (HgS), mercury selenide (HgSe), mercury telluride (HgTe), zinc oxide (ZnO), cadmium oxide (CdO), mercury oxide (HgO), cadmium selenium sulfide (CdSeS), cadmium selenium telluride (CdSeTe), cadmium sulfide telluride (CdSTe), cadmium zinc sulfide (CdZnS), cadmium zinc selenide (CdZnSe), cadmium sulfide selenide (CdSSe), cadmium zinc telluride (CdZnTe), cadmium mercury sulfide (CdHgS), cadmium mercury selenide (CdHgSe), cadmium mercury telluride (CdHgTe), zinc selenium sulfide (ZnSeS), zinc selenium telluride (ZnSeTe), zinc sulfide telluride (ZnSTe), mercury selenium sulfide (HgSeS), mercury selenium telluride (HgSeTe), mercury sulfide telluride (HgSTe), mercury zinc sulfide (HgZnS), mercury zinc selenide (HgZnSe), cadmium zinc oxide (CdZnO), cadmium mercury oxide (CdHgO), zinc mercury oxide (ZnHgO), zinc selenium oxide (ZnSeO), zinc tellurium oxide (ZnTeO), zinc sulfide oxide (ZnSO), cadmium selenium oxide (CdSeO), cadmium tellurium oxide (CdTeO), cadmium sulfide oxide (CdSO), mercury selenium oxide (HgSeO), mercury tellurium oxide (HgTeO), mercury sulfide oxide (HgSO), cadmium zinc selenium sulfide (CdZnSeS), cadmium zinc selenium telluride (CdZnSeTe), cadmium zinc sulfide telluride (CdZnSTe), cadmium mercury selenium sulfide (CdHgSeS), cadmium mercury selenium telluride (CdHgSeTe), cadmium mercury sulfide telluride (CdHgSTe), mercury zinc selenium sulfide (HgZnSeS), mercury zinc selenium telluride (HgZnSeTe), mercury zinc sulfide telluride (HgZnSTe), cadmium zinc selenium oxide (CdZnSeO), cadmium zinc tellurium oxide (CdZnTeO), cadmium zinc sulfide oxide (CdZnSO), cadmium mercury selenium oxide (CdHgSeO), cadmium mercury tellurium oxide (CdHgTeO), cadmium mercury sulfide oxide (CdHgSO), zinc mercury selenium oxide (ZnHgSeO), zinc mercury tellurium oxide (ZnHgTeO) and zinc mercury sulfide oxide (ZnHgSO), but the present disclosure is not limited thereto.

In one aspect of the present disclosure, the Group 13 to 15-element-based compounds may include one or more of gallium phosphide (GaP), gallium arsenide (GaAs), gallium antimonide (GaSb), gallium nitride (GaN), aluminum phosphide (AlP), aluminum arsenide (AlAs), aluminum antimonide (AlSb), aluminum nitride (AlN), indium phosphide (InP), indium arsenide (InAs), indium antimonide (InSb), indium nitride (InN), gallium phosphide arsenide (GaPAs), gallium phosphide antimonide (GaPSb), gallium phosphide nitride (GaPN), gallium arsenide nitride (GaAsN), gallium antimonide nitride (GaSbN), aluminum phosphide arsenide (AlPAs), aluminum phosphide antimonide (AlPSb), aluminum phosphide nitride (AlPN), aluminum arsenide nitride (AlAsN), aluminum antimonide nitride (AlSbN), indium phosphide arsenide (InPAs), indium phosphide antimonide (InPSb), indium phosphide nitride (InPN), indium arsenide nitride (InAsN), indium antimonide nitride (InSbN), aluminum gallium phosphide (AlGaP), aluminum gallium arsenide (AlGaAs), aluminum gallium antimonide (AlGaSb), aluminum gallium nitride (AlGaN), aluminum arsenide nitride (AlAsN), aluminum antimonide nitride (AlSbN), indium gallium phosphide (InGaP), indium gallium arsenide (InGaAs), indium gallium antimonide (InGaSb), indium gallium nitride (InGaN), indium arsenide nitride (InAsN), indium antimonide nitride (InSbN), aluminum indium phosphide (AlInP), aluminum indium arsenide (AlInAs), aluminum indium antimonide (AlInSb), aluminum indium nitride (AlInN), aluminum arsenide nitride (AlAsN), aluminum antimonide nitride (AlSbN), aluminum phosphide nitride (AlPN), gallium aluminum phosphide arsenide (GaAlPAs), gallium aluminum phosphide antimonide (GaAlPSb), gallium indium phosphide arsenide (GaInPAs), gallium indium aluminum arsenide (GaInAlAs), gallium aluminum phosphide nitride (GaAlPN), gallium aluminum arsenide nitride (GaAlAsN), gallium aluminum antimonide nitride (GaAlSbN), gallium indium phosphide nitride (GaInPN), gallium indium arsenide nitride (GaInAsN), gallium indium aluminum nitride (GaInAlN), gallium antimonide phosphide nitride (GaSbPN), gallium arsenide phosphide nitride (GaAsPN), gallium arsenide antimonide nitride (GaAsSbN), gallium indium phosphide antimonide (GaInPSb), gallium indium phosphide nitride (GaInPN), gallium indium antimonide nitride (GaInSbN), gallium phosphide antimonide nitride (GaPSbN), indium aluminum phosphide arsenide (InAlPAs), indium aluminum phosphide nitride (InAlPN), indium phosphide arsenide nitride (InPAsN), indium aluminum antimonide nitride (InAlSbN), indium phosphide antimonide nitride (InPSbN), indium arsenide antimonide nitride (InAsSbN) and indium aluminum phosphide antimonide (InAlPSb), but the present disclosure is not limited thereto.

In one aspect of the present disclosure, the Group 14 to 16 element-based compounds may include one or more of tin oxide (SnO), tin sulfide (SnS), tin selenide (SnSe), tin telluride (SnTe), lead sulfide (PbS), lead selenide (PbSe), lead telluride (PbTe), germanium oxide (GeO), germanium sulfide (GeS), germanium selenide (GeSe), germanium telluride (GeTe), tin selenium sulfide (SnSeS), tin selenium telluride (SnSeTe), tin sulfide telluride (SnSTe), lead selenium sulfide (PbSeS), lead selenium telluride (PbSeTe), lead sulfide telluride (PbSTe), tin lead sulfide (SnPbS), tin lead selenide (SnPbSe), tin lead telluride (SnPbTe), tin oxide sulfide (SnOS), tin oxide selenide (SnOSe), tin oxide telluride (SnOTe), germanium oxide sulfide (GeOS), germanium oxide selenide (GeOSe), germanium oxide telluride (GeOTe), tin lead sulfide selenide (SnPbSSe), tin lead selenium telluride (SnPbSeTe) and tin lead sulfide telluride (SnPbSTe), but the present disclosure is not limited thereto.

In one aspect of the present disclosure, the water soluble ligand present in the water soluble ligand layer may be one or more selected from the group consisting of silica, polyethylene glycol (PEG), polyethylenimine (PEI), mercaptopropionic acid (MPA), cysteamine, mercapto-acetic acid, mercapto-undecanol, 2-mercapto-ethanol, 1-thio-glycerol, deoxyribonucleic acid (DNA), mercapto-undecanoic acid, 1-mercapto-6-phenyl-hexane, 1,16-dimecapto-hexadecane, 18-mercapto-octadecyl amine, tri-octyl phosphine, 6-mercapto-hexane, 6-mercapto-hexanoic acid, 16-mercapto-hexadecanoic acid, 18-mercapto-octadecyl amine, 6-mercapto-hexyl amine, 8-hydroxy-octylthiol, 1-thio-glycerol, mercapto-acetic acid, mercapto-undecanoic acid, hydroxamate, hydroxamic acid derivatives, ethylene diamine, glutathione, N-acetylcysteine, thioctic acid, tiopronin, mercaptosuccinic acid, dithiothreitol, dihydrolipoic acid and bucillamine, but the present disclosure is not limited thereto.

In one aspect of the present disclosure, the quantum dot may consist of CdSe and ZnS.

In one aspect of the present disclosure, the average diameter of the quantum dot may be 1 to 50 nm, and specifically, 1 to 30 nm or 1 to 20 nm. Here, the average diameter of the quantum dot may correspond to the range of all integers present in the above range. Specifically, the average diameter of the quantum dot may be 1 nm or more, 2 nm or more, 3 nm or more, 4 nm or more, 5 nm or more, 6 nm or more, 7 nm or more, 8 nm or more, 9 nm or more, 10 nm or more, 15 nm or more, and may be 50 nm or less, 40 nm or less, 35 nm or less, 30 nm or less, 25 nm or less, 20 nm or less, 19 nm or less, 18 nm or less, 17 nm or less, 16 nm or less, 15 nm or less, 14 nm or less, 13 nm or less, 12 nm or less, 11 nm or less or 10 nm or less.

In one aspect of the present disclosure, the average diameter of the quantum dot bead may be 50 nm to 2 μm. Here, the average diameter of the quantum dot bead may correspond to the range of all integers present in the above range. Specifically, the average diameter of the quantum dot bead may be 50 nm or more, 100 nm or more, 120 nm or more, 140 nm or more, 160 nm or more, 180 nm or more, 200 nm or more, 250 nm or more, 300 nm or more, 400 nm or more, 450 nm or more, 500 nm or more, 700 nm or more, 900 nm or more or 1 μm or more, and may be 2 μm or less, 1.5 μm or less, 1 μm or less, 900 nm or less, 800 nm or less, 750 nm or less, 700 nm or less, 650 nm or less, 600 nm or less, 550 nm or less, 500 nm or less, 450 nm or less, 400 nm or less, 350 nm or less or 300 nm or less. When the average diameter of the quantum dot bead is more than 1 μm, it is inappropriate to use the quantum dot bead because the beads are difficult to move when used in a lateral flow sensor.

In one aspect of the present disclosure, the antigen may be one or more selected from the group consisting of a C-reactive protein (CRP), influenza, malaria, hepatitis C virus (HCV), human immunodeficiency virus (HIV), hepatitis B virus (HBV), creatine kinase MB (CK-MB), troponin I, myoglobin, prostate specific antigen (PSA), alpha-fetoprotein (AFP), carcinoembryonic antigen (CEA), thyroid stimulating hormone (TSH), chorionic somatomammotropin hormone (CSH), human chorionic gonadotropin (hCG), cortisol, progesterone, and testosterone.

In one aspect of the present disclosure, the first antibody may be one or more selected from the group consisting of a polyclonal anti-CRP antibody, a polyclonal anti-influenza antibody, a polyclonal anti-malaria antibody, a polyclonal anti-HCV antibody, a polyclonal anti-HIV antibody, a polyclonal anti-HBV antibody, a polyclonal anti-CK-MB antibody, a polyclonal anti-troponin I antibody, a polyclonal anti-myoglobin antibody, a polyclonal anti-PSA antibody, a polyclonal anti-AFP antibody, a polyclonal anti-CEA antibody, a polyclonal anti-TSH antibody, a polyclonal anti-CSH antibody, a polyclonal anti-hCG antibody, a polyclonal anti-cortisol antibody, a polyclonal anti-progesterone antibody, and a polyclonal anti-testosterone antibody.

In one aspect of the present disclosure, the second antibody may be one or more selected from the group consisting of a monoclonal anti-CRP antibody, a monoclonal anti-influenza antibody, a monoclonal anti-malaria antibody, a monoclonal anti-HCV antibody, a monoclonal anti-HIV antibody, a monoclonal anti-HBV antibody, a monoclonal anti-CK-MB antibody, a monoclonal anti-troponin I antibody, a monoclonal anti-myoglobin antibody, a monoclonal anti-PSA antibody, a monoclonal anti-AFP antibody, a monoclonal anti-CEA antibody, a monoclonal anti-TSH antibody, a monoclonal anti-CSH antibody, a monoclonal anti-hCG antibody, a monoclonal anti-cortisol antibody, a monoclonal anti-progesterone antibody, and a monoclonal anti-testosterone antibody.

In one aspect of the present disclosure, the biological sample may be one or more selected from the group consisting of urine, blood, serum, plasma and saliva, but the present disclosure is not limited thereto.

In one aspect of the present disclosure, an immunochromatographic detection method for a target antigen in a biological sample, which includes: (a) injecting a biological sample into a first inlet; (b) binding a target antigen in the sample to a linker having a first antibody in a conjugate pad while the injected biological sample is developed; (c) binding the antigen-linker complex to a second antibody immobilized in a test area; (d) injecting a quantum dot bead having the second antibody into a second inlet; and (e) binding the quantum dot bead to the antigen-linker complex present in the test area while the quantum dot bead is developed, may be provided.

In one aspect of the present disclosure, an immunochromatographic detection method for a target antigen in a biological sample, which includes: (a) injecting a biological sample into a first inlet; (b) binding a target antigen in the sample with a linker having a first antibody by passing the same through a linker pad while the injected biological sample is developed; (c) binding the antigen-linker complex to a second antibody immobilized in a test area; (d) injecting a buffer solution into a second inlet or releasing the buffer solution into a quantum dot pad by breaking a container having the buffer solution by an external force; and (e) moving a quantum dot bead having the second antibody to the test area while the buffer solution is developed, and binding the quantum dot bead to the antigen-linker complex present in the test area, may be provided.

In one aspect of the present disclosure, the immunochromatographic detection method may further include (f) measuring the fluorescence of the quantum dot bead by irradiating the test area with UV light after Step (e).

In one aspect of the present disclosure, the immunochromatographic detection method may further include washing the test area before Step (d). The washing step may be washing an unreacted material (e.g., an antigen and an antigen-linker complex) in the test area.

In one aspect of the present disclosure, a method of diagnosing a target antigen-related disease, disorder or condition, which uses the immunochromatographic detection method according to one aspect of the present disclosure and further includes determining a patient's condition with respect to the target antigen from the measured fluorescence detection data, may be provided.

In one aspect of the present disclosure, a method of amplifying the fluorescence detection intensity or sensitivity of a biosensor using a quantum dot bead, which includes: bringing a linker having a first antibody into contact with an antigen in a biological sample; bringing a quantum dot bead having a second antibody into contact with the antigen-linker complex; and forming an antigen-linker-quantum dot bead sandwich structure, may be provided.

In one aspect of the present disclosure, a lateral flow immunosensor using the immunochromatographic detection method according to one aspect of the present disclosure may be provided.

In one aspect of the present disclosure, a bio-diagnostic apparatus for detecting a biological material, which includes: a linker pad including a linker having a first antibody; a quantum dot bead pad including a quantum dot bead having a second antibody; a test pad having a test area in which the second antibody is immobilized; and an absorbent pad connected with the test pad, may be provided. In one aspect of the present disclosure, the absorbent pad may impart a capillary force to allow a fluid (e.g., a sample and a buffer solution) to be developed. In one aspect of the present disclosure, the fluid may move to the absorbent pad by pressure.

In one aspect of the present disclosure, the bio-diagnostic apparatus may further include a light irradiation unit configured to irradiate the test area with light. In one aspect of the present disclosure, the light irradiation unit may emit UV light, and directly irradiates the test area present in the test pad with light such as UV light. In one aspect of the present disclosure, the light irradiation unit may be present at a location which does not interfere with the fluid flow on the test pad, and the light irradiation may be performed with the range, intensity and time that does not interfere with the antigen-antibody reaction in the test area. The light irradiation unit may facilitate easy confirmation of the antigen-antibody reaction in the test area, and induces the fluorescence of the quantum dot bead in the test area. Accordingly, the presence or absence of the target antigen may be measured/detected.

In one aspect of the present disclosure, the bio-diagnostic apparatus may further include a first inlet into which a biological sample of a subject for detecting a physiological material is input, and a second inlet into which a buffer solution is input or a buffer solution container containing a buffer solution.

In one aspect of the present disclosure, the buffer solution container may contain a buffer solution, and may release the buffer solution into the quantum dot bead pad when broken by an external force. In one aspect of the present disclosure, the buffer solution container may be broken by an external force when the buffer solution should be developed to the quantum dot bead pad, and then the buffer solution is released from the container. The external force refers to, for example, the pressure by a finger or any type of force applied by a structure or means for breaking the buffer solution container. In one aspect of the present disclosure, the buffer solution container may be present at the end of a quantum dot bead membrane channel or washing membrane channel.

In one aspect of the present disclosure, the biological sample input into the first inlet may pass through the linker pad, at this time, the target antigen present in the biological sample may react with/bind to the linker present in the linker pad, the resulting antigen-linker complex may react with/bind to the second antibody present in the test area again, and thus the second antibody-antigen-linker complex may be formed in the test area. In one aspect of the present disclosure, the buffer solution input through the second inlet or the buffer solution developed from the buffer solution container may pass through the quantum dot bead pad, the quantum dot bead may reach the test area with the buffer solution to react with/bind to the second antibody-antigen-linker complex, and thus the second antibody-antigen-linker-quantum dot bead complex may be formed in the test area.

In one aspect of the present disclosure, the linker pad may be connected with the first inlet, and the quantum dot bead pad may be connected with the second inlet or buffer solution container. Here, “connected” may mean that the buffer solution allowing the sample or quantum dot bead to move is input or injected through each inlet or using the container such that the sample or quantum dot bead is positioned to pass through the linker pad or quantum dot bead pad.

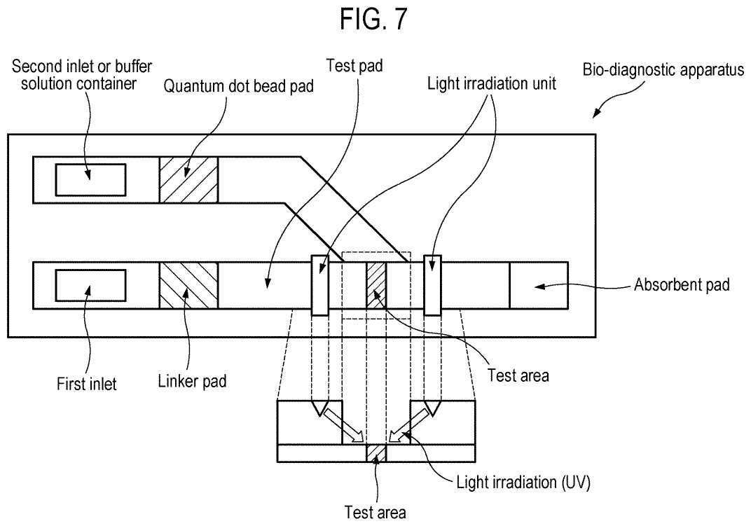

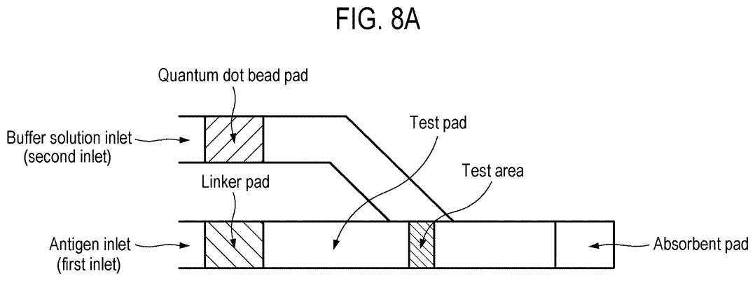

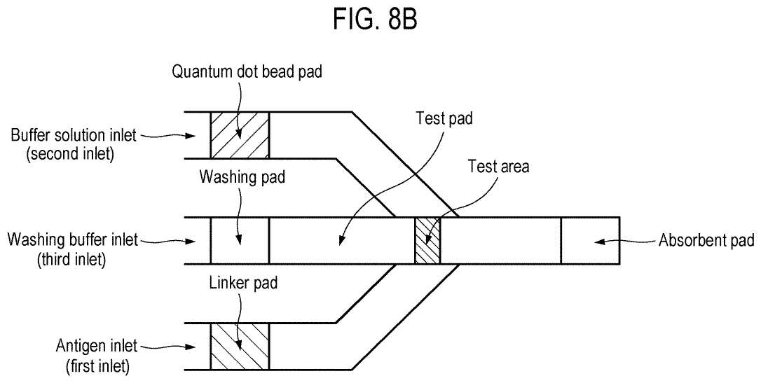

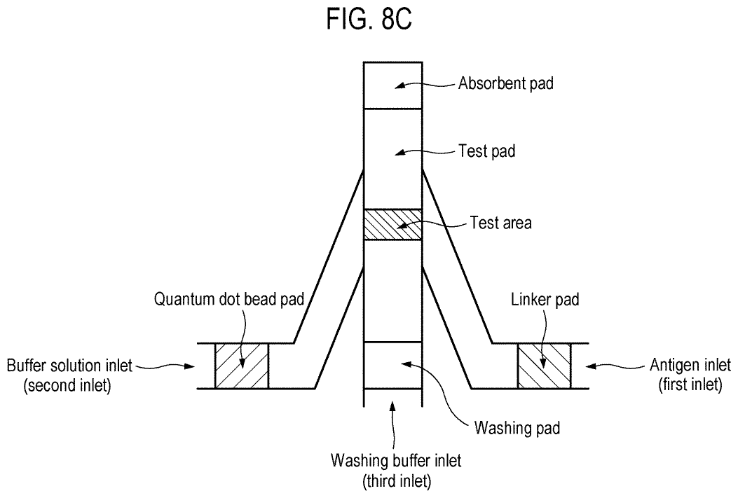

In one aspect of the present disclosure, the first inlet and the linker pad may be present in a linker membrane channel, the second inlet or buffer solution container and the quantum dot bead pad may be present in the quantum dot bead membrane channel, and the test area is present in the linker membrane channel, wherein the linker membrane channel and the quantum dot bead membrane channel may meet in the test area. The linker membrane channel and the quantum dot bead membrane channel may be formed or aligned to allow the quantum dot bead to be easily developed to the test area as shown in FIGS. 7 and 8A to 8C. Here, each membrane channel may refer to a unit structure in which a fluid such as a buffer solution that allows the sample or quantum dot bead to move flows.

In one aspect of the present disclosure, the linker membrane channel may further include a sample pad connecting the first inlet with the linker pad, and the quantum dot bead membrane channel may further include a buffer solution pad connecting the second inlet or buffer solution container with the quantum dot bead pad. In one aspect of the present disclosure, in the bio-diagnostic apparatus, the first inlet may be formed to expose the linker pad to the outside, and the sample may be input into the exposed linker pad through the first inlet. In one aspect of the present disclosure, in the bio-diagnostic apparatus, the second inlet may be formed to expose the buffer solution pad to the outside, and the buffer solution may be input into the exposed buffer solution pad through the second inlet.

In one aspect of the present disclosure, the inlet or the buffer solution container present in each membrane channel (e.g., the first inlet of the linker membrane channel, the second inlet or buffer solution container of the quantum dot bead membrane channel, and a third inlet or washing buffer container of a washing membrane channel) may be present at the ends of the same side or opposite sides of the bio-diagnostic apparatus.

In one aspect of the present disclosure, in each membrane channel (e.g., the linker membrane channel, the quantum dot bead membrane channel, and the washing membrane channel), the flow of fluids such as the biological sample and the buffer solution may be in the same or different direction, and the fluid flows may be directed to the absorbent pad. Specifically, in one aspect of the present disclosure, when each membrane channel includes one absorbent pad, the fluid flows may be in the same direction, and when each membrane channel includes a separate absorbent pad, the fluid flow may be in the same or different direction.

In one aspect of the present disclosure, the biological sample may reach the test area earlier than the quantum dot bead moving along with the buffer solution.

In one aspect of the present disclosure, the first inlet may be closer to the test area than the second inlet or buffer solution container, so that the biological sample may reach the test area earlier than the quantum dot bead. In one aspect of the present disclosure, the linker membrane channel and the quantum dot bead membrane channel may have different lengths, and due to such difference, the biological sample may bind to the linker having the first antibody and to the second antibody immobilized in the test area, and then the quantum dot bead moving along with the buffer solution may reach the test area and bind to the antigen-linker complex.

In one aspect of the present disclosure, the absorbent pad may be present as one in all of the membrane channels present in the bio-diagnostic apparatus (e.g., the linker membrane channel, the quantum dot bead membrane channel, and the washing membrane channel), or be present separately in each membrane channel.

In one aspect of the present disclosure, the membrane channels may be divided into separate chambers.

In one aspect of the present disclosure, the pads included in the bio-diagnostic apparatus may have a stacked structure.

In one aspect of the present disclosure, the bio-diagnostic apparatus may further include a third inlet into which a washing buffer is input or a washing buffer container, and a washing pad connected with the third inlet or washing buffer container. In one aspect of the present disclosure, the third inlet or washing buffer container and the washing pad may be present in the washing membrane channel. Here, the washing buffer may wash a substance (antigen, linker or quantum dot bead, etc.) which does not participate in a reaction but is present in the test area. In addition, the washing buffer container may be the same as the buffer solution container, but may allow the buffer solution for washing the test area, not the quantum dot bead, to be developed. In one aspect of the present disclosure, the washing membrane channel may meet another membrane channel in the test area, and for example, may be formed or aligned as shown in FIGS. 7 and 8A to 8C.

In one aspect of the present disclosure, the bio-diagnostic apparatus may be a lateral flow immunosensor, but the present disclosure is not limited thereto.

The bio-diagnostic apparatus or lateral flow immunosensor according to one aspect of the present disclosure may have a schematic diagram as shown in FIG. 7.

In one aspect of the present disclosure, the membrane channels present in the bio-diagnostic apparatus or lateral flow immunosensor and the pads included therein may have schematic diagrams as shown in FIGS. 8A to 8C.

In one aspect of the present disclosure, a bio-diagnostic kit including the bio-diagnostic apparatus of the present disclosure, and a buffer solution container including a buffer solution may be provided.

Hereinafter, the configuration and effects of the specification will be described in further detail with reference to examples and experimental examples. However, these examples and comparative examples are merely provided to help in the understanding of the specification, and the scope and range of the specification are not limited to the following examples.

### [Preparation Example 1] Preparation of Quantum Dot Having an Antibody on its Surface

(1) Preparation of Lipid Soluble Quantum Dot

In a 3-neck flask, 1.0 g of zinc acetate (Zn(Ac)₂), 0.441 g of cadmium oxide (CdO), 20 mL of oleic acid and 75 mL of octadecene (ODE) were mixed, and water was removed at 150° C. for 1 hour under a nitrogen atmosphere. Subsequently, the resulting flask was heated to 300° C., and then 1 mL of trioctylphosphine (TOP) and 0.045 g of selenium (Se) were injected and heated for 3 minutes, thereby forming the core of a quantum dot.

Subsequently, 0.5 mL of dodecanethiol was added to the 3-neck flask and reacted for 10 minutes. A solution containing 1 mL of TOP and 0.025 g of sulfur (S) was then added to the reaction vessel of the 3-neck flask and reacted for 20 minutes, thereby forming a shell. Then, the resulting core and shell were purified with a mixed solution of ethanol and toluene and dissolved in an organic solvent, thereby obtaining a primary quantum dot.

0.5 g of the resulting primary quantum dots, 1 g of zinc acetate, 0.21 g of cadmium oxide, 10 mL of oleic acid and 35 mL of octadecene were put into a separate 3-neck flask, and reacted at 300° C. for 30 minutes. Subsequently, 0.5 mL of octanethiol was added and stirred for 10 minutes, a solution containing 1 mL of TOP and 0.025 g of sulfur was put into the reaction vessel of the 3-neck flask and reacted for 20 minutes. Afterward, the resulting mixture was purified with a mixed solution of ethanol and toluene and dissolved in an organic solvent, thereby obtaining a secondary quantum dot. Such a quantum dot has a core-stable layer-shell-lipid soluble ligand layer structure.

(2) Preparation of Carboxyl Group-Substituted Water Soluble Quantum Dots

20 mg of the secondary quantum dots were added to a reaction vessel containing 1 mL mercaptopropionic acid (MPA) and reacted at 60° C. for 60 minutes, thereby obtaining the final quantum dots having a water soluble ligand (carboxyl group).

(3) Preparation of PEI-Substituted Water Soluble Quantum Dots

PEI was mixed with tetrahydrofuran (“THF”), thereby resulting in an 80 mg/mL PEI solution.

0.25 μL of the secondary quantum dots of Preparation Example 1-(1) having a concentration of 10 mg/mL was mixed with 400 μL of THF, 500 μL of the PEI-THF solution was slowly added thereto, and then reacted at room temperature overnight. Subsequently, the resulting product was purified with THF and dissolved in distilled water, thereby preparing quantum dots (PEI-quantum dot) having an amine group.

(4) Preparation of Quantum Dots Having an Antibody

The quantum dots of Preparation Example 1-(1), 1-ethyl-3-(3-dimethylaminopropyl)carbodiimide (EDC) and N-hydroxysuccinimide (NHS) were mixed, and reacted for 2 hours at room temperature. After the reaction, the resulting mixture was centrifuged, washed with tertiary distilled water three times, and a polyclonal anti-CRP antibody (Invitrogen Corp.) was added to have a concentration 5-fold higher than that of the quantum dots (based on mole number), followed by a reaction for 1 hour at room temperature. After the reaction, the resulting product was centrifuged, washed with tertiary distilled water three times, treated with bovine serum albumin (BSA), followed by a reaction for 1 hour at room temperature.

After the reaction, the resulting product was centrifuged, washed with tertiary distilled water three times, and stored by dispersing in a 1 ml solution containing a 1M Tris buffer (pH 8), 0.1% Tween 20 and 0.1% Triton-X-100.

### [Preparation Example 2] Preparation of Quantum Dot Beads Having an Antibody on its Surface

(1) Synthesis of a Silica Particle Substrate and Surface Modification

A silica-based nanoparticle was synthesized by the Stober process. First, NH₄OH, EtOH and H₂O were stirred in a ratio of 3:60:1 mL in an Erlenmeyer flask, 2 mL of tetraethyl orthosilicate (TEOS) was added to the reactants, and the mixture was stirred at 50° C. and reacted for 18 hours or more. Here, the reaction time and the mixing ratio may be adjusted according to the desired size. Subsequently, a final sample was obtained by a centrifuge using ethanol. Here, approximately 200 nm of silica beads may be obtained.

Subsequently, for a reaction between the surface and the quantum dots, as reactive functional groups, 180 μL each of 3-mercaptopropyltrimethoxysilane (MPTS) and NH₄OH were added, and reacted for 12 to 24 hours. After purification by a centrifuge using ethanol, a surface-modified silica particle substrate was obtained.

(2) Binding Quantum Dots to the Substrate

The ratio of the quantum dots of Preparation Example 1-(1) and the surface-modified silica substrate was 50:100 (mg), chloroform was added at twice the volume of the above mixture and then stirred, followed by a reaction for 30 minutes. After the reaction, a quantum dot bead was obtained.

(3) Surface Modification of Quantum Dot Beads

The CdSe/ZnS quantum dot beads synthesized in Preparation Example 2-(2) and MPA (50 mg: 20 μL) were mixed with chloroform and ethanol (2 mL:2 mL) and reacted by mixing for 10 hours, and a water soluble ligand, which is a carboxyl group, was attached to the outer surface of the final quantum dot bead to modify the surface, followed by purification using ethanol and a centrifuge.

(4) Preparation of Quantum Dot Beads Having an Antibody

100 nmol of EDC and NHS was respectively dispersed in 30 μL PBS (pH 5, 100 nmol/30 μL).

In a 1.5 mL tube containing 0.1 nmol of the quantum dot beads (—COOH) synthesized in Preparation Example 2-(3), 30 μL of the EDC and NHS respectively dispersed in PBS were added, and reacted with a vortex for 2 hours.

After the reaction, the quantum dot beads (—COOH) were spun down through centrifugation and dispersed in 150 μL of PBS. Subsequently, a monoclonal or polyclonal anti-CRP antibody (Invitrogen Corp.) was added to have a concentration (based on mole number) 10-fold higher than that of the quantum dot beads (—COOH), and reacted for 1 hour.

After the reaction, the resulting product was washed with Tween 20 phosphate buffered saline (TPBS) twice and PBS (pH 7.4) once. Subsequently, the resulting product was dispersed in 1 mL of 5% BSA, and reacted with a vortex for 1 hour. After the reaction, the resulting product was washed with TPBS twice and PBS (pH 7.4) once. Finally, the resulting product was dispersed in a 1 ml solution containing 1M Tris buffer (pH 8), 0.1% Tween 20 and 0.1% Triton X-100 and stored.

### [Experimental Example 1] Confirmation of the Characteristics of Quantum Dots and Quantum Dot Beads

(1) The Zeta Potential of Quantum Dots and the Quantum Efficiency of Quantum Dots and Quantum Dot Beads

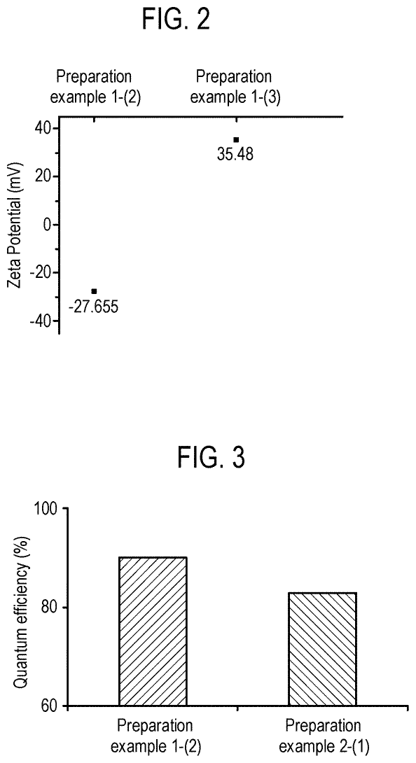

The zeta potentials of the quantum dots of Preparation Examples 1-(2) and 1-(3) were measured using ELS-100ZS (Otsuka), and the result is shown in FIG. 2.

The quantum efficiency of the quantum dots of Preparation Example 1-(2) and the quantum dot beads of Preparation Example 2-(3) was measured using QE 2000 (Otsuka Corp.), and the result is shown in FIG. 3. According to these results, the quantum dots and the quantum dot beads, which were prepared in Preparation Examples 1-(2) and 2-(3) according to one aspect of the present disclosure, exhibited a quantum efficiency of 92±3% and 83±3%, respectively, and as both are over 80%, excellent effects are exhibited.

(3) Confirmation of the Sizes and Shapes of Quantum Dots and Quantum Dot Beads

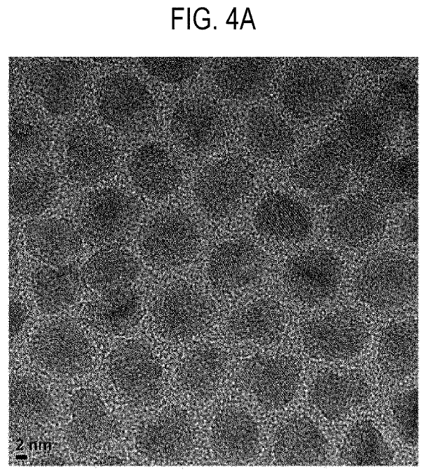

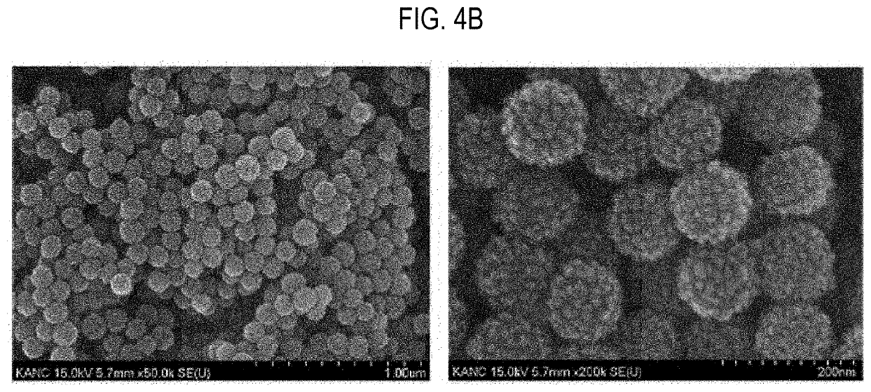

To determine the sizes and shapes of the quantum dots of Preparation Example 1-(1) and the quantum dot beads of Preparation Example 2-(2), JEM-2100F (JEOL Ltd.) and FE-SEM (Hitachi Corp.) were used, and a transmission electron micrograph of the quantum dots is shown in FIG. 4A, and a scanning electron micrograph of the quantum dot beads is shown in FIG. 4B. According to these results, it can be confirmed that both the quantum dots and the quantum dot beads according to one aspect of the present disclosure have a spherical shape with a uniform size.

(4) Particle Size Analysis of Quantum Dot Beads

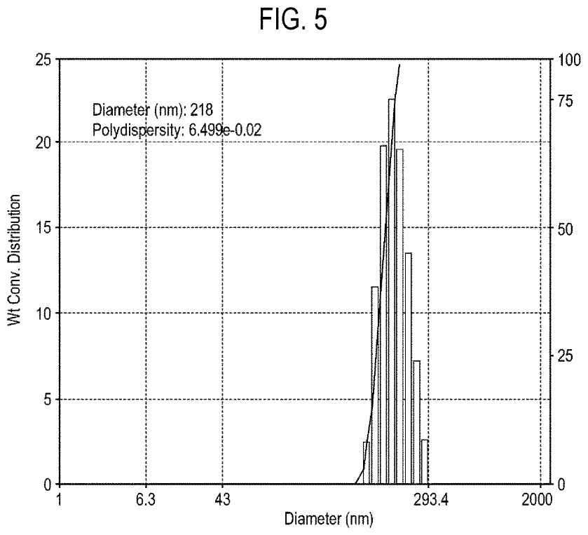

A particle size analysis of the quantum dot beads of Preparation Example 2-(2) was performed using ELS100 (Otsuka Corp.), and the result is shown in FIG. 5. From the result, it can be confirmed that the quantum dot beads that can be used herein exhibit high polydispersity. When nano-fluorescent substances agglomerate, the efficiency may deteriorate and the problem of non-specific noise may occur. Therefore, whether or not the original size is maintained is an important factor in the use of the beads as a fluorescent substance. Since the quantum dot beads that can be used herein exhibit high polydispersity, the above-mentioned problem may not occur.

### [Experimental Example 2] Experiment to Confirm the Fluorescent Reactivity in Lateral Flow Immunosensor

### Comparative Examples 1 and 2

3 pmol (1 μL) of a monoclonal anti-CRP antibody (Invitrogen Corp.) was injected into a nitrocellulose (NC) membrane test area of a biosensor and then dried. In Comparative Example 1, the quantum dots of Preparation Example 1-(4) binding to a polyclonal anti-CRP antibody, and in Comparative Example 2, the quantum dot beads of Preparation Example 2-(4) binding to a polyclonal anti-CRP antibody were injected into a conjugate pad and then dried.

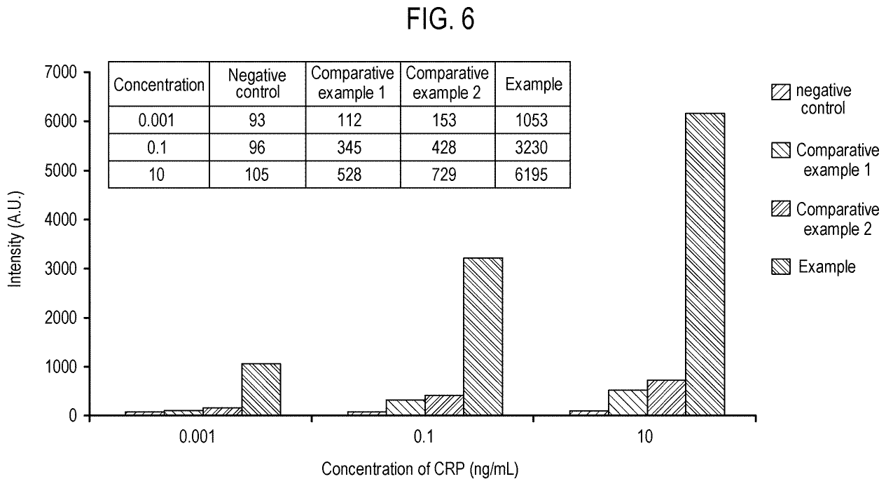

A CRP antigen (0.001 ng/mL, 0.1 ng/mL or 10 ng/mL; Invitrogen Corp.) was put into a first inlet and developed for 5 minutes. After the development, the fluorescence intensity of the biosensor was measured using a QD-J7 fluorescent analyzer, and the result is shown in the graph in FIG. 6.

### Example

3 pmol (1 μL) of a monoclonal anti-CRP antibody (Invitrogen Corp.) was injected into a NC membrane test area of a biosensor and then dried. The quantum dot of Preparation Example 1-(4) binding to a polyclonal anti-CRP antibody was injected into a conjugate pad and then dried.

A CRP antigen (0.001 ng/mL, 0.1 ng/mL or 10 ng/mL; Invitrogen Corp.) was put into a first inlet and developed for 5 minutes, the quantum dot bead of Preparation Example 2-(4) binding to the monoclonal anti-CRP antibody was put into a second inlet, and the solution developed for 10 minutes. After the development, the fluorescence intensity of the biosensor was measured using a QD-J7 fluorescent analyzer, and the result is shown in the graph of FIG. 6.

As per FIG. 6, according to a detection method using a quantum dot (linker) and a quantum dot bead according to one aspect of the present disclosure, it was confirmed that, in all antigen concentration ranges, sensitivity or fluorescence intensity for detecting an antigen is exhibited at least 10-fold higher than when a quantum dot (linker) and a quantum dot bead are individually used.

Compared to the individually used quantum dot, this result shows that the quantum dot bead exhibits a higher fluorescence, and the detection intensity is significantly amplified. Compared to the quantum dot of Comparative Example 1, since the quantum dot bead of Comparative Example 2 contains at least 200 to 500-fold more quantum dots, the fluorescence detection intensity or detection sensitivity should correspondingly increase. However, the result shown in FIG. 6 is actually similar to that using the quantum dot of Comparative Example 1. This is because the number of first antibodies (e.g., polyclonal anti-CRP antibodies) binding to one quantum dot bead increases similarly to the increasing number of quantum dots, resulting in a reduction in the number of detected antigens and a decrease in detection intensity. On the other hand, according to the method of the present disclosure, it was indicated that the quantum dot as a linker first binds to an antigen so as to increase the number of antigens contributing to fluorescence detection, and then binds to the quantum dot bead so as to very significantly amplify the detection intensity without the loss of the antigens participating in the detection.

In addition, when the quantum dot bead is individually used, due to the large size of the bead, before binding to the immobilized second antibody, the bead is probably swept away along the flow of the sample, whereas when the quantum dot bead binds to the antigen using a linker, compared to individual use, the quantum dot bead can more stably bind to an antigen, indicating that the detection intensity is very significantly amplified.

As above, as specific parts of the specification have been described in detail, although it is clear to those skilled in the art that this specific technique is merely a preferred embodiment, the scope of the specification is not limited thereto. Thus, a substantial scope of the specification will be defined by the accompanying claims and their equivalents.

1. An immunochromatographic detection method for a target antigen in a biological sample, comprising:
binding a linker having a first antibody to a quantum dot bead having a second antibody through a target antigen,
wherein the first antibody and the second antibody are specific for different sites of the target antigen.

2. The method of claim 1, wherein the linker forms a complex by binding to the antigen before binding to the quantum dot bead.

3. The method of claim 1, wherein the linker is a material that is capable of binding to an antibody.

4. The method of claim 3, wherein the linker is one or more selected from the group consisting of quantum dots, colloidal gold nanoparticles, colloidal carbon, colloidal selenium, up-conversion fluorescent nanoparticles, europium (III) chelate microparticles, dye-doped nanoparticles, magnetic nanoparticles, electroactive nanoparticles, silica, alumina, titanium dioxide, zinc dioxide, polystyrene, and polymethylmethacrylate.

5. The method of claim 4, wherein the linker is a quantum dot.

6. The method of claim 5, wherein the quantum dot included in the quantum dot bead and the quantum dot, which is the linker, have a core-stable layer-shell-water soluble ligand layer structure.

7. The method of claim 6, wherein the core includes one or more of cadmium (Cd) and selenium (Se),
the stable layer includes one or more of cadmium (Cd), selenium (Se), zinc (Zn) and sulfur (S), and
the shell includes one or more of cadmium (Cd), selenium (Se), zinc (Zn) and sulfur (S).

8. The method of claim 1, wherein the quantum dot included in the quantum dot bead and the quantum dot, which is the linker, include one or more of Group 12 to 16 element-based compounds, Group 13 to 15-element-based compounds and Group 14 to 16 element-based compounds.

9. The method of claim 8, wherein the Group 12 to 16 element-based compounds include one or more of cadmium sulfide (CdS), cadmium selenide (CdSe), cadmium telluride (CdTe), zinc sulfide (ZnS), zinc selenide (ZnSe), zinc telluride (ZnTe), mercury sulfide (HgS), mercury selenide (HgSe), mercury telluride (HgTe), zinc oxide (ZnO), cadmium oxide (CdO), mercury oxide (HgO), cadmium selenium sulfide (CdSeS), cadmium selenium telluride (CdSeTe), cadmium sulfide telluride (CdSTe), cadmium zinc sulfide (CdZnS), cadmium zinc selenide (CdZnSe), cadmium sulfide selenide (CdSSe), cadmium zinc telluride (CdZnTe), cadmium mercury sulfide (CdHgS), cadmium mercury selenide (CdHgSe), cadmium mercury telluride (CdHgTe), zinc selenium sulfide (ZnSeS), zinc selenium telluride (ZnSeTe), zinc sulfide telluride (ZnSTe), mercury selenium sulfide (HgSeS), mercury selenium telluride (HgSeTe), mercury sulfide telluride (HgSTe), mercury zinc sulfide (HgZnS), mercury zinc selenide (HgZnSe), cadmium zinc oxide (CdZnO), cadmium mercury oxide (CdHgO), zinc mercury oxide (ZnHgO), zinc selenium oxide (ZnSeO), zinc tellurium oxide (ZnTeO), zinc sulfide oxide (ZnSO), cadmium selenium oxide (CdSeO), cadmium tellurium oxide (CdTeO), cadmium sulfide oxide (CdSO), mercury selenium oxide (HgSeO), mercury tellurium oxide (HgTeO), mercury sulfide oxide (HgSO), cadmium zinc selenium sulfide (CdZnSeS), cadmium zinc selenium telluride (CdZnSeTe), cadmium zinc sulfide telluride (CdZnSTe), cadmium mercury selenium sulfide (CdHgSeS), cadmium mercury selenium telluride (CdHgSeTe), cadmium mercury sulfide telluride (CdHgSTe), mercury zinc selenium sulfide (HgZnSeS), mercury zinc selenium telluride (HgZnSeTe), mercury zinc sulfide telluride (HgZnSTe), cadmium zinc selenium oxide (CdZnSeO), cadmium zinc tellurium oxide (CdZnTeO), cadmium zinc sulfide oxide (CdZnSO), cadmium mercury selenium oxide (CdHgSeO), cadmium mercury tellurium oxide (CdHgTeO), cadmium mercury sulfide oxide (CdHgSO), zinc mercury selenium oxide (ZnHgSeO), zinc mercury tellurium oxide (ZnHgTeO) and zinc mercury sulfide oxide (ZnHgSO).

10. The method of claim 8, wherein the Group 13 to 15-element-based compounds include one or more of gallium phosphide (GaP), gallium arsenide (GaAs), gallium antimonide (GaSb), gallium nitride (GaN), aluminum phosphide (AlP), aluminum arsenide (AlAs), aluminum antimonide (AlSb), aluminum nitride (AlN), indium phosphide (InP), indium arsenide (InAs), indium antimonide (InSb), indium nitride (InN), gallium phosphide arsenide (GaPAs), gallium phosphide antimonide (GaPSb), gallium phosphide nitride (GaPN), gallium arsenide nitride (GaAsN), gallium antimonide nitride (GaSbN), aluminum phosphide arsenide (AlPAs), aluminum phosphide antimonide (AlPSb), aluminum phosphide nitride (AlPN), aluminum arsenide nitride (AlAsN), aluminum antimonide nitride (AlSbN), indium phosphide arsenide (InPAs), indium phosphide antimonide (InPSb), indium phosphide nitride (InPN), indium arsenide nitride (InAsN), indium antimonide nitride (InSbN), aluminum gallium phosphide (AlGaP), aluminum gallium arsenide (AlGaAs), aluminum gallium antimonide (AlGaSb), aluminum gallium nitride (AlGaN), aluminum arsenide nitride (AlAsN), aluminum antimonide nitride (AlSbN), indium gallium phosphide (InGaP), indium gallium arsenide (InGaAs), indium gallium antimonide (InGaSb), indium gallium nitride (InGaN), indium arsenide nitride (InAsN), indium antimonide nitride (InSbN), aluminum indium phosphide (AlInP), aluminum indium arsenide (AlInAs), aluminum indium antimonide (AlInSb), aluminum indium nitride (AlInN), aluminum arsenide nitride (AlAsN), aluminum antimonide nitride (AlSbN), aluminum phosphide nitride (AlPN), gallium aluminum phosphide arsenide (GaAlPAs), gallium aluminum phosphide antimonide (GaAlPSb), gallium indium phosphide arsenide (GaInPAs), gallium indium aluminum arsenide (GaInAlAs), gallium aluminum phosphide nitride (GaAlPN), gallium aluminum arsenide nitride (GaAlAsN), gallium aluminum antimonide nitride (GaAlSbN), gallium indium phosphide nitride (GaInPN), gallium indium arsenide nitride (GaInAsN), gallium indium aluminum nitride (GaInAlN), gallium antimonide phosphide nitride (GaSbPN), gallium arsenide phosphide nitride (GaAsPN), gallium arsenide antimonide nitride (GaAsSbN), gallium indium phosphide antimonide (GaInPSb), gallium indium phosphide nitride (GaInPN), gallium indium antimonide nitride (GaInSbN), gallium phosphide antimonide nitride (GaPSbN), indium aluminum phosphide arsenide (InAlPAs), indium aluminum phosphide nitride (InAlPN), indium phosphide arsenide nitride (InPAsN), indium aluminum antimonide nitride (InAlSbN), indium phosphide antimonide nitride (InPSbN), indium arsenide antimonide nitride (InAsSbN) and indium aluminum phosphide antimonide (InAlPSb).

11. The method of claim 8, wherein the Group 14 to 16 element-based compounds include one or more of tin oxide (SnO), tin sulfide (SnS), tin selenide (SnSe), tin telluride (SnTe), lead sulfide (PbS), lead selenide (PbSe), lead telluride (PbTe), germanium oxide (GeO), germanium sulfide (GeS), germanium selenide (GeSe), germanium telluride (GeTe), tin selenium sulfide (SnSeS), tin selenium telluride (SnSeTe), tin sulfide telluride (SnSTe), lead selenium sulfide (PbSeS), lead selenium telluride (PbSeTe), lead sulfide telluride (PbSTe), tin lead sulfide (SnPbS), tin lead selenide (SnPbSe), tin lead telluride (SnPbTe), tin oxide sulfide (SnOS), tin oxide selenide (SnOSe), tin oxide telluride (SnOTe), germanium oxide sulfide (GeOS), germanium oxide selenide (GeOSe), germanium oxide telluride (GeOTe), tin lead sulfide selenide (SnPbSSe), tin lead selenium telluride (SnPbSeTe) and tin lead sulfide telluride (SnPbSTe).

12. The method of claim 8, wherein the quantum dot included in the quantum dot bead and the quantum dot, which is the linker, consist of CdSe and ZnS.

13. The method of claim 1, wherein the linker has an average diameter of 1 to 300 nm.

14. (canceled)

15. The method of claim 1, wherein the quantum dot bead has an average diameter of 50 nm to 2 μm.

16. (canceled)

17. The method of claim 5, wherein the quantum dot has an average diameter of 1 to 50 nm.

18. The method of claim 1, wherein the antigen is one or more selected from the group consisting of a C-reactive protein (CRP), influenza, malaria, hepatitis C virus (HCV), human immunodeficiency virus (HIV), hepatitis B virus (HBV), creatine kinase MB (CK-MB), troponin I, myoglobin, prostate specific antigen (PSA), alpha-fetoprotein (AFP), carcinoembryonic antigen (CEA), thyroid stimulating hormone (TSH), chorionic somatomammotropin hormone (CSH), human chorionic gonadotropin (hCG), cortisol, progesterone, and testosterone.

19. The method of claim 1, wherein the first antibody is one or more selected from the group consisting of a polyclonal anti-CRP antibody, a polyclonal anti-influenza antibody, a polyclonal anti-malaria antibody, a polyclonal anti-HCV antibody, a polyclonal anti-HIV antibody, a polyclonal anti-HBV antibody, a polyclonal anti-CK-MB antibody, a polyclonal anti-troponin I antibody, a polyclonal anti-myoglobin antibody, a polyclonal anti-PSA antibody, a polyclonal anti-AFP antibody, a polyclonal anti-CEA antibody, a polyclonal anti-TSH antibody, a polyclonal anti-CSH antibody, a polyclonal anti-hCG antibody, a polyclonal anti-cortisol antibody, a polyclonal anti-progesterone antibody, and a polyclonal anti-testosterone antibody.

20. The method of claim 1, wherein the second antibody is one or more selected from the group consisting of a monoclonal anti-CRP antibody, a monoclonal anti-influenza antibody, a monoclonal anti-malaria antibody, a monoclonal anti-HCV antibody, a monoclonal anti-HIV antibody, a monoclonal anti-HBV antibody, a monoclonal anti-CK-MB antibody, a monoclonal anti-troponin I antibody, a monoclonal anti-myoglobin antibody, a monoclonal anti-PSA antibody, a monoclonal anti-AFP antibody, a monoclonal anti-CEA antibody, a monoclonal anti-TSH antibody, a monoclonal anti-CSH antibody, a monoclonal anti-hCG antibody, a monoclonal anti-cortisol antibody, a monoclonal anti-progesterone antibody, and a monoclonal anti-testosterone antibody.

21. The method of claim 1, wherein the biological sample is selected from the group consisting of urine, blood, serum, plasma and saliva.

22-25. (canceled)

26. A method of diagnosing a target antigen-related disease, disorder or condition using the immunochromatographic detection method according to claim 1, further comprising:
determining a patient's condition with respect to the target antigen from the measured fluorescence detection data.

27. A lateral flow immunosensor, which uses the detection method according to claim 1.

28-43. (canceled)
